(19) United States
(12) Patent Application Publication
(10) Pub. No.: US 2006/0248663 A1
(43) Pub. Date: Nov. 9, 2006

(54) Compositions comprising discrete particles aggregates and discrete particle agglomerates for application to keratin fibers

(75) Inventors: Mario Elmen Tremblay, West Chester, OH (US); Vladimir Gartstein, Cincinnati, OH (US); Rolanda Jeanette Johnson, Liberty Township, OH (US); Robert Wayne Glenn, Virginia Water (GB)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company

(21) Appl. No.: 11/414,088

(22) Filed: Apr. 28, 2006

Related U.S. Application Data
(60) Provisional application No. 60/677,438, filed on May 3, 2005.

(51) Int. Cl. A61K 8/00

(52) U.S. Cl. 8/405

(57) ABSTRACT

Discrete particle aggregates and/or discrete particle agglomerates for application onto keratin fibers, preferably hair, optionally combined with one or more oxidative and/or direct dyes.

### CROSS REFERENCE TO RELATED APPLICATIONS

This application claims the benefit of U.S. Provisional application Ser. No. 60/677,438 (Case 9981P), filed on May 3, 2005.

### FIELD OF THE INVENTION

The present invention relates to compositions comprising one or more discrete particle aggregates and/or agglomerates, and methods of using said compositions for application onto keratin fibers, preferably hair.

### BACKGROUND OF THE INVENTION

Changing the properties of hair by the deposition of actives onto the surface of the hair is well known. The purpose of the actives can be to improve the volume of the hair, improve its combability or improve its shine and appearance. In addition, these actives can be used to change the colour of the hair. However, many such actives are deposited on only the surface of the hair and can affect the feel of the hair giving it a rough texture. In addition, the actives deposited on surface of the hair often quickly wash off or rub off during wear. This is especially an issue if the purpose is to colour the hair and obtain good coloring and wearability without undesirable side effects to the hair and skin. One approach to colouring the hair is the use of oxidative dyes but many such compounds result in colorations that demonstrate poor wearability and stability to light, and some have recently been associated with negative effects from a toxicology point of view.

Another recent alternative to the deposition of actives onto hair has been the use of nano (10⁻⁹)-sized particles. This alternative has generally relied upon the employment of discrete particles, provided in single particle form that are <2 nm in size. However, it remains technically very difficult and burdensome to prevent individual discrete particles from aggregating and/or agglomerating to form larger particles during processing and/or formulation.

Nanoparticles have been described in the art. For example .WO01/45652 describes the use of nanonscale hair colorants for the production of hair colourant preparations by the use of rapid expansion of supercritical solutions (RESS). However this method is technically very difficult and burdensome. US2004/0010864 describes organo modified metallic nanoparticles in suspension form to dye or treat human hair. EP1440681 describes the use of luminescent semiconductive nanoparticles capapble of emitting radiation at 400-700 nm under excitation with light.

However, here still remains a need in the art to deliver consumer benefits such as shine, hold, body, sheen, volume, stiffness, weight, curl and condition in an efficient manner. There also remains a need in the art to provide alternatives to conventional dyes and compositions for dyeing of hair comprising them. Said alternatives should provide one or more of the following benefits when applied to hair: intense and even coloration, shade and color stability over a reasonable period of time (good wash fastness, constancy upon exposure to light, and/or acid perspiration), low skin staining and a favourable safety profile. It has now been surprisingly found the use of compositions comprising the defined discrete particle aggregates and/or agglomerates, both alone and in combination with oxidative and/or direct dyes, resolves the problems associated with the use of particles in the conventional manner.

### SUMMARY OF THE INVENTION

This invention relates to a composition comprising one or more discrete particle aggregates and/or agglomerates for application to keratin fibers, preferably hair. Indeed, it has been surprisingly discovered, and documented via the present disclosure, that the reduced size of the discrete particle aggregates and/or agglomerates of the present invention can deliver the defined benefits without the previously observed negatives. The particle aggregates and agglomerates of the present invention further comprise a chromophoric material selected from the group consisting of: metal oxides, aluminum, ceramic, cerium, copper, diamond, gold, graphite, hasteloy, indium, platinum, silicon, silver, talc, tin, zinc and zirconium carbon black, gold colloid, silver colloid, metal nano-composites, non-metal nano-composites, doped metal oxides, synthetic or naturally occurring melanin (or derivatives), organic pigments and mixtures thereof. In one aspect of the present invention, the discrete particle aggregates and discrete particle agglomerates of the present invention may have a size of from about 2 nanometers to about 1000 nanometers, preferably from about 10 nanometers to about 500 nanometers, more preferably from about 20 nanometers to about 400 nanometers, most preferably from about 20 nanometers to about 200 nanometers. In another aspect of the present invention, the compositions disclosed herein may be combined with oxidative and/or direct dyes. The present invention further seeks to encompass hair care products comprising said compositions and methods of using both the present hair care products and compositions to induce one or more physical and/or chemical changes to the keratin fibers to which they are applied.

### DETAILED DESCRIPTION OF THE INVENTION

Except as otherwise noted, amounts represent approximate weight percent of the actual amount of the ingredient, and do not include solvents, fillers or other materials which may be combined with the ingredient in commercially available products, and the amounts include the composition in the form of intended use. Except as otherwise noted, all amounts including part, percentages, and proportions are understood to be modified by the word “about”, and amounts are not intended to indicate significant digits.

As used herein, the term “hair” refers to keratinous fibers on a living, e.g. a person, or non-living body, e.g. in a wig, hairpiece, or other aggregation of non-living keratinous fibers. Mammalian, preferably human, hair is a preferred. Notably, hair, wool, fur, and other keratinous fibers are suitable substrates for coloring by the compounds and compositions described herein.

As used herein, the term ‘hair composition’ refers to a composition used on the hair to deliver the desired chemical or physical benefit.

As used herein, the term “hair dyeing composition” refers to the composition containing one or more oxidation dyes, including the compounds described herein, prior to admixture with the developer composition. The term “developer composition” (which encompasses the term oxidizing agent composition) refers to compositions containing an oxidizing agent prior to admixture with the hair dyeing composition. The term “hair dyeing system” refers to the combination of the hair dyeing composition and the developer composition before admixture, and may further include a conditioner product and instructions, such product or system often being provided packaged as a kit. The term “hair dyeing product composition” refers to the composition formed by mixing the hair dyeing composition and the developer composition.

As used herein, “cosmetically acceptable” means that ingredients which the term describes are suitable for use in contact with the skin or hair of humans and lower animals without undue toxicity, incompatibility, instability, irritation, allergic response, and the like.

Discrete Particle Aggregates and Agglomerates

In one aspect of the present invention, there are provided compositions for application to keratin fibers, preferably hair, comprising one or more discrete particle aggregates and/or discrete particle agglomerates. The discrete particle aggregates and/or agglomerates of the present invention comprise a chromophoric material. It has been surprisingly discovered, and documented via the present disclosure, that the reduced size of the discrete particle of the present invention can regulate the uptake and retention of hair composition actives particularly dyes by the keratin fibers to which they are applied. Without wishing to be bound by theory, it is believed that the increased uptake and retention of the subject discrete particle aggregates and discrete particle agglomerates is attributable to the specified size range of the agglomerates and/or aggregates. If the particle size is less than 2 nm it can penetrate through the skin and the hair which would provide a safety risk. The primary particles, before aggregation and/or agglomeration, are in the usual case less than 2 nm in size. If the particle size is greater than 1000 nm the hair would have a poor hair feel and in addition the particle could readily transfer off the hair making the benefit short-lived. It has been surprisingly identified that the selection of discrete particles having a particle size as claimed allow the chromophoric material to penetrate the penetrate the hair cuticle structure thereby ensuring its resistance ot being washed out or otherwise physically removed form the cuticle. In one aspect of the present invention, the compositions may comprise discrete particle aggregates, discrete particle agglomerates and combinations thereof. In yet still another aspect of the present invention the dyeing compositions disclosed herein may further comprise one or more oxidative and/or direct dyes.

In one aspect of the present invention the discrete particle aggregate and discrete particle agglomerates disclosed herein comprise one or more chromophoric materials selected from the group consisting of: alumina; aluminium; aluminum nitride; aluminum oxide (alpha & gamma); antimony pentoxide; antimony tin oxide; barium sulfate; barium titanate; brass; calcium carbonate; calcium chloride; calcium oxide; carbon black; ceramic; ceria; samarium doped; cerium; cerium oxide; chromium oxide; cobalt; cobalt oxide; copper; copper oxide; copper oxide; custom chemistry; diamond, dispersions; doping of nanoparticles; dysprosium oxide; erbium oxide; europium oxide; ferric oxide; amorphous; fluorescent; gadolinium oxide; gold; graphite; hafnium oxide; hastelloy; hematite- (alpha, beta, amorphous, epsilon, and gamma); indium; indium oxide; indium tin oxide; iron, iron-cobalt alloy; iron-nickel alloy; iron oxide; iron oxide, Fe203; iron oxide, Fe304; iron oxide, transparent; iron sulphide; lanthanum; lanthanum oxide, lead; lead oxide; lithium manganese oxide; lithium titanate; lithium vanadium oxide; lubricant grades, spherical; luminescent; magnesia; magnesium; magnesium oxide; magnetic nanoparticles; magnetite; manganese oxide; molybdenum; molybdenum oxide; montmorillonite clay; nanodots; nanometals; nano oxide suspensions; neodymium oxide; nickel; niobia; niobium; niobium oxide; palladium; platinum; platinum—silver; praseodymium oxide; ruthenium; silica; silicon; silicon carbide, (beta & amorphous); silicon dioxide; silicon nitride, (alpha & amorphous); silicon nitride- yttrium oxide; silicon nitride- yttrium oxide- aluminum oxide; silver; specialty; stainless steel; talc; tantalum; terbium oxide; tin; tin oxide; titania; titanium; titanium carbide; titanium diboride; titanium dioxide, anatase grade; titanium dioxide, rutile grade; titanium nitride; titanium oxide; tungsten; tungsten carbide- cobalt; tungsten oxide; vanadium oxide; yellow iron oxide; yttria; yttria stabilized zirconia; yttrium; yttrium oxide; zinc; zinc oxide; zinc sulfide; zirconia; zirconium; zirconium dioxide; zirconium oxide; zirconium silicate; ceramic nanopowders; nanocomposite particles and combinations thereof. Other chromophoric materials suitable for use in the context of the present discrete particle aggregates and/or discrete particle agglomerates are commercially-available from BASF Corporation; Nanophase Technologies Corporation; Nyacol Nano Technologies; Nanotechnologies Inc; Nanoscale; KOBO Inc; Cadre Co.; Tokai Chemicals; Cabot Company; US Cosmetics Inc.; Merck and Degussa.

Preferably the discrete particle aggregate and discrete aggolomerate particles are selected from the group consisting of metal oxides, aluminum, ceramic, cerium, copper, diamond, gold, graphite, hasteloy, indium, platinum, silicon, silver, talc, tin, zinc and zirconium carbon black, gold colloid, silver colloid, metal nano-composites, non-metal nano-composites, synthetic or naturally occurring melanin and derivatives, organic pigments and mixtures thereof. The metal oxides are selected from the group consisting of: aluminum oxide, zinc oxide, cerium oxide, copper oxide, silicon oxide, zirconium oxide, titanium oxide, niobium oxide, iron oxide, doped metal oxides and combinations thereof.

The discrete particle aggregates and agglomerates of the present invention are not only intended for use on keratin fibers. In one aspect of the present invention, the discrete particle aggregates and discrete particle agglomerates may be used on any porous material, preferably a porous biological material. Non-limiting examples of suitable porous materials in the context of the present invention may include skin, hair, woven fabrics and non-wovens. In another aspect of the present invention, the discrete particle aggregates and/or agglomerates disclosed herein may be adapted to change a physical characteristic of a keratin fiber to which they are applied. Non-limiting examples of physical characteristics that application of the discrete particle aggregates and/or agglomerates of the present invention may induce include color, shine, hold, body, sheen, volume, stiffness, weight, curl, condition, water evaporation, water repellence, thickness, strength and mixtures thereof. In another aspect of the present invention, the discrete particle aggregates and/or discrete particle agglomerates of the present may be adapted to induce a chemical change in the keratin fibers to which they are applied. Non-limiting examples of chemical changes which application of the present compositions may induce include: activation of an oxidizing species; deactivation of an oxidizing species; activation of a color forming species; deactivation a color forming species; scavenging of free radicals; boosting of the efficacy of oxidizing species; changing the HLB balance of the hair resulting in altered uptake of organic and/or inorganic species subsequently applied and combinations thereof. In yet still another aspect of the present invention, the discrete particle aggregates and/or agglomerates disclosed herein may be adapted to change both a physical and chemical characteristic of the keratin fibers to which they are applied.

The precise size of the discrete particle aggregates and discrete particle agglomerates of the present invention will depend upon several factors including the needs and/or abilities of the formulator and the nature of the keratin fibers onto which application of the present compositions is intended. Those skilled in the art to which the subject invention pertains will appreciate that there exist numerous apparatuses and methods for determining the size of the aggregates and agglomerates of the present invention and can be readily selected by the skilled person depending on the nature of the formulation into which the discrete particles are to be incorporated. For example for aqueous compositions a preferred method utilises a Particle Size Analyzer. One such apparatus for use for particle size analysis is the Horiba Laser Scattering and Particle Size and Distribution Analyzer (model # LA-930) from the Horiba Company.

Another such apparatus is a Malvern particle size instrument (model ZetaNanoSizer S with a 633 nm HeNe laser) manufactured by Malvern Instruments Ltd. The Malvern particle size instrument is particularly useful in that it can be used to determine the discrete particle size of aqueous formulations and emulsions. The Malvern particle size instrument can determine a particle size range from about 2 nm to about 100 micron. Samples are taken from the top, middle and bottom, of the composition to ensure homogenous mixing of the particles through the formulation and repeat samples are measured to ensure a repeatable particle size is obtained. In the majority of cases a distribution of particle sizes is achieved.

In one aspect of the present invention, discrete particle aggregates and/or agglomerates comprising one or more of the above-mentioned chromophoric materials are provided. An aggregate is composed of partially fused, reasonably spherical primary particles, where the primary particle is the smallest possible particle that is formed in the manufacturing process. By “aggregates,” it is intended that the at least two primary particles are in edge- or surface- contact with one another. The aggregates are held together by attractive Van der Waals forces to form agglomerates. The Van der Waals forces increase as the size of the primary particle is reduced and the agglomerate density is increased.

Without wishing to be bound by theory it is believed that the aggregates and/or agglomerates of the primary particles are generally formed during the manufacturing process and that these aggregates and/or agglomerates are of a particle size greater than 1000 nm. There are several methods of producing such materials, including fume particles, plasma particles and sol-gel. The fumed process involves converting a starting compound to the gas phase and then reacting it spontaneously and quantitatively in an oxyhydrogen flame with the intermediate-formed water to produce the desired particle. During this chemical reaction a considerable amount of heat is released, which is eliminated in a cooling line. By varying the concentration of the co-reactants, the flame temperature, and the dwell time of the silica in the combustion chamber, is it possible to influence the particle size. The plasma process involves vaporizing materials in a hot plasma zone and cooling them in a way that controls both the structure and chemistry of the powder. For sol-gel techniques, the starting materials used in the preparation of the “sol” are usually inorganic metal salts or metal organic compounds such as metal alkoxides. In a typical sol-gel process, the precursor is subjected to a series of hydrolysis and polymerazation reactions to form a colloidal suspension, or a “sol”. Further processing of the “sol” enables one to make ceramic materials in different forms. Thin films can be produced on a piece of substrate by spin-coating or dip-coating. When the “sol” is cast into a mold, a wet “gel” will form. With further drying and heat-treatment, the “gel” is converted into dense ceramic or glass articles. If the liquid in a wet “gel” is removed under a supercritical condition, a highly porous and extremely low density material called “aerogel” is obtained. As the viscosity of a “sol” is adjusted into a proper viscosity range, ceramic fibers can be drawn from the “sol”. Ultra-fine and uniform ceramic powders are formed by precipitation, spray pyrolysis, or emulsion techniques. Other references that describe ways in which to synthesize the discrete particle aggregates and agglomerates include: Nanoparticles and Nanostructured Films: Preparation, Characterization and Applications (J. H. Fendler, Ed.) John Wiley & Son Ltd, 1998; Friedlander, S. K. Synthesis of Nanoparticles and Their Agglomerates: Aerosol Reactors. WTEC Hyper Librarian. 1998; Friedlander, S. K. 1977. Smoke, dust and haze: Fundamentals of aerosol behavior. New York: Wiley Interscience; Siegel, R. W. 1994. Nanophase materials: Synthesis, structure and properties. In Physics of new materials, ed. F. E. Fujita. Berlin, Germany: Springer-Verlag; Grandqvist, C. G., and R. A. Buhrman. 1976. Ultrafine metal particles. J. Appl. Phys. 47: 2200; Gurav, A., T. Kodas, T. Pluym, and Y. Xiong. 1993. Aerosol processing of materials. Aerosol Sci. Technol. 19: 411; Marijnissen, J. C. M., and S. Pratsinis, eds. 1993. Synthesis and measurement of ultrafine particles. Delft Univ. Press.; Nagel, S. R., J. B. MacChesney, and K. L. Walder. 1985. Modified chemical vapor deposition. In Optical fiber communications, vol. 1, ed. Li Tingye. Academic Press; Pratsinis, S. E., and S. V. R. Mastrangelo. 1989. Material synthesis in aerosol reactors. Chem. Eng. Prog. 85(5): 62; Pratsinis, S. E., and T. T. Kodas. 1993. Manufacturing of materials by aerosol processes; In Aerosol measurement, ed. K. Willeke and P. A. Baron. New York: Van Nostrand Reinhold; Ulrich, G. D. 1984. Flame synthesis of fine particles. Chem. Eng. News 62: 22; Windeler, R. S., and S. K. Friedlander. 1997. Production of nanometer-sized metal oxide particles by gas phase reactions in a free jet. I. Experimental system and results. In press, Aerosol Sci. Technol; Windeler, R. S., K. E. J. Lehtinen, and S. K. Friedlander. 1997. Production of nanometer-sized metal oxide particles by gas phase reaction in a free jet. II. Particle size and neck formation—Comparison with theory. Aerosol Sci. Technol. In press; Wu, M. K., R. S. Windeler, C. K. R. Steiner, T. Bors, and S. K. Friedlander. 1993. Controlled synthesis of nanosized particles by aerosol processes. Aerosol Sci. Technol. 19: 527; Friedlander, S. K., H. D. Jang, and K. H. Ryu. 1997. Elastic behavior of nanoparticle chain aggregates. Submitted for publication; Karch, J., R. Birringer, and H. Gleiter. 1987. Ceramics ductile at low temperatures. Nature. 330: 556-58; Schleicher, B., and S. K. Friedlander. 1996. Fabrication of aerogel-like structures by agglomeration of aerosol particles in an electric field. J. Colloid Interface Sci. 180: 15-21; Siegel, R. W. 1994. Nanophase materials: Synthesis, structure and properties. In Physics of new materials, ed. F. E. Fujita. Berlin, Germany: Springer-Verlag.

The subject aggregate and/or agglomerate may possess a size of from about 2 nanometers to about 1000 nanometers, preferably from about 10 nanometers to about 500 nanometers, more preferably from about 20 nanometers to about 400 nanometers, most preferably from about 20 nanometers to 200 nanometers. Preferably the discrete particles of the present invention have a mean particle size of from about 2 nanometers to about 1000 nanometers, preferably from about 10 nanometers to about 500 nanometers, more preferably from about 20 nanometers to about 400 nanometers, most preferably from about 20 nanometers to 200 nanometers.

In another aspect of the present invention, at least about 95%, preferably at least about 98%, more preferably at least about 99% of the discrete particle aggregates and/or agglomerates of the present compositions may possess a size of about 2 nanometers to about 1000 nanometers, preferably from about 10 nanometers to about 500 nanometers, more preferably from about 20 nanometers to about 400 nanometers, most preferably from about 20 nanometers to 200 nanometers. In another aspect of the present invention, at least about 95%, preferably at least about 98%, more preferably at least about 99% of the discrete particle aggregates and/or agglomerates of the present compositions may possess a size of greater than about 20 nanometers. It has been found that the use of discrete particles of the present invention having a narrow particle size distribution is particularly advantageous and result in the need for reduced concentration of the discrete particles in order to achieve the same benefit.

In yet another aspect of the present invention, discrete particle agglomerates comprising one or more of the above-described chromophoric materials are provided. By “agglomerates,” it is intended that two or more primary particles are in point-contact with one another. Without whishing to be bound by theory, it is believed that any two individual discrete particles may combine to form an agglomerate in accordance with the present invention. When two or more discrete (or primary) particles combine to form an agglomerate in accordance with the present invention, the subject agglomerate may possess a size of from about 2 nanometers to about 1000 nanometers, preferably from about 10 nanometers to about 500 nanometers, more preferably from about 20 nanometers to about 400 nanometers, most preferably from about 20 nanometers to about 200 nanometers. In another aspect of the present invention, at least about 95% of the discrete particle agglomerates of the present compositions may possess a size of less than about 200 nanometers. In another aspect of the present invention, at least about 95% of the discrete particle agglomerates of the present compositions may possess a size of greater than about 20 nanometers.

Several methods are particularly useful to disperse and deagglomerate the discrete particles so that they have the required size. An effective means of deagglomerating and dispersing are required to overcome the bonding forces after wetting the discrete particles. For this application, ultrasound has proven to be an effective method compared to many other methods such as rotor stator mixers, piston homogenizers, gear pumps or wet grinding methods, such as beat mills, colloid mills and ball mills. Dispersion by ultrasound is a consequence of microturbulences caused by fluctuation of pressure and cavitation, which is the formation, growth, and impulsive collapse of bubbles in a liquid. Investigations using different materials, such as aqueous solutions of discrete particulate iron oxide powder agglomerates, have demonstrated the considerable advantage of ultrasound when compared with other technologies. For certain applications milling of the discrete particles prior to ultrsonication can also be advantageous.

In yet another aspect of the present invention, the discrete particle aggregates and/or agglomerates of the present invention may be provided in hydrophobic form. The primary size of the hydrophobic discrete particle aggregates and/or agglomerates of the present invention may be from about 2 nanometers to about 1000 nanometers, preferably from about 10 nanometers to about 500 nanometers, more preferably from about 20 nanometers to about 400 nanometers, most preferably from about 20 nanometers to about 200 nanometers. In yet another aspect of the present invention, the discrete particle aggregates and/or agglomerates of the present invention may be provided in hydrophilic form. The primary size of the hydrophilic discrete particles of the present invention is from 2 nanometers to about 1000 nanometers, preferably from about 10 nanometers to about 500 nanometers, more preferably from about 20 nanometers to about 400 nanometers. The hydrophilic form of the present discrete particles aggregates and/or agglomerates is particularly useful in conveying one or more antimicrobial benefits. The hydrophobicity and/or hydrophillicity characteristics of the present discrete particle aggregates and/or agglomerates may be altered using techniques known in the art, including but not limited to surface treatment.

In yet another aspect of the present invention, the surface structures of the discrete particle aggregates and/or agglomerates disclosed herein may possess separations of from about 0 to about 10 particle diameters, preferably from about 2 to about 8 particle diameters, more preferably from about 4 to about 6 particle diameters. In yet still another aspect of the present invention, the discrete particle aggregates and/or agglomerates disclosed herein may possess surface structures that are formed by particles, or by particle fractions that have differing particle sizes or particle diameters. In one aspect of the present invention, the surface structure may have at least two particle fractions whose average particle size differs from a factor of from about 2 to about 10, preferably by a factor of from about 4 to about 8.

The present discrete particle aggregates and agglomerates, as well as the individual discrete particles that constitute them, of the present compositions may take a variety of shapes depending on the needs and/or abilities of the formulator and the nature of the keratin fibers onto which application of the present compositions is intended. Suitable shapes for the discrete particle aggregates and agglomerates of the present invention may include: spheres, flakes, needles, plates and combinations thereof. The use of rod-shaped or sphere-shaped discrete particle aggregates and/or agglomerates is preferred, sphere-shaped discrete particles are particularly preferred. In another aspect of the present invention, the discrete particle aggregates and agglomerates of the present invention may possess an irregular fine nanostructure on the surface. In one aspect, the fine structure of the discrete particle aggregates and agglomerates may take the form of a fissured structure with elevations and/or depressions provided in the nanometer range. In one aspect, the average height of the elevations may be from about 1 nanometer to about 100 nanometers, preferably from about 5 nanometers to about 50 nanometers, more preferably from about 10 nanometers to about 30 nanometers. In yet still another aspect of the present invention, the elevations and/or depressions of the discrete particle aggregates and/or agglomerates is less than about 500 nanometers, preferably less than about 200 nanometers, more preferably less than about 100 nanometers. Without wishing to be bound by theory, it is believed the presence of depressions, craters, crevices, notches, clefts, apertures or cavities on the surface of the present discrete particle aggregates and/or agglomerates serves to reinforce their overall structure. In yet another aspect of the present invention, the discrete particles aggregates and/or agglomerates disclosed herein may comprise other structure features such as undercuts in the depressions or combinations of various depressions.

In yet still another aspect of the present invention, the discrete particle aggregates and/or agglomerates, and the discrete particles that constitute them, may be provided in an encapsulated form. In one aspect, the discrete particle aggregates and/or agglomerates may be encapsulated by core shells. In another aspect of the present invention, said encapsulates may further comprise an organic or inorganic colored material. Suitable organic colored materials include nanoparticles based on polymers or biological macromolecules, for example nanoparticles of 1,3-diphenyl-5-(2-anthryl)-2-pyrazoline (DAP), polyvinyl nanoparticles, polystyrene nanoparticles (polymer colloids), proteins, nanoparticles based on interactions of surfactants with oppositely charged polymers, dendritic polymer particles, polymer-clay particles and combinations thereof. Suitable methods for encapsulating the discrete particle aggregates and/or agglomerates in accordance with the present invention are described in Huang, Q., and Y. Jiang. 2004, Enhancing the stability of phenolic antioxidants by nanoencapsulatio, 228th American Chemical Society National Meeting. August 21-26. Philadelphia; Jang, S.-Y., M. Marquez, and G. A. Sotzing. 2004. Rapid direct nanowriting of conductive polymer via electrochemical oxidative nanolithography, Journal of the American Chemical Society 126:9476; Leser, M. E., M. Michel, and H. J. Watzke. 2003. Food goes nano—New horizons for food structure research. In Food Colloids, Biopolymers and Materials. E. Dickinson and T. Van Vliet, eds. Cambridge, England: Royal Society of Chemistry; Loscertales, I. G. M. Marquez, et al. 2002. Micro/nano encapsulation via electrified coaxial liquid jets, Science 295:1695; Moraru. C. I., Q. Huang, et al. 2003. Nanotechnology: A new frontier in food science, Food Technology 57:24; Ruengruglikit, C. and Q. Huang. 2004. Fabrication of nanoporous oligonucleotide microarray for pathogen detection and identification, 227th American Chemical Society National Meeting. March 28-April 1, Anaheim; Dinsmore, A. D. and D. A. Weitz. 2002. Colloidosomes: Selectively permeable capsules composed of colloidal particles, Science 298:1006; Sotzing, G. A., et al. 2002. Preparation and properties of vapor detector arrays formed from poly(3,4-ethylenedioxy) thiophene-poly(styrene sulfonate)/insulating polymer composites, Analytical Chemistry 72:3181; Ogawa S., Decker E. A., McClements D. J., 2004, “Production and characterization of O/W emulsions containing droplets stabilized by lecithin-chitosan-pectin mutilayered membranes,” J. Agr. Food Chem. 52 (11): 3600; Ogawa S., Decker E. A., McClements D. J., 2003. Influence of environmental conditions on the stability of oil in water emulsions containing droplets stabilized by lecithin-chitosan membranes, J. Agr. Food Chem. 51 (18): 5522. Customized encapsulation services that may be useful in accordance with the present invention are commercially-available from NanosominÔ Serum, from Elsom Research Co., LivOn Labs, Inc., Nanophase Technologies, MCC SA (Switzerland), Advanced Nano-Products, Inc.

In yet another aspect of the present invention, one or more retention methods may be applied to lock the present discrete particle aggregates and/or agglomerates into the keratin fiber to which they are applied. These retention methods may include the application of light, application of temperature, change in pH, application of solvents, surface treatment, application of dispersants and/or combinations thereof. Without wishing to be bound by theory, it is believed that the application of one or more of the above retention methods results in the creation of chemical bonds between the subject discrete particle aggregates and agglomerates and the subject keratin fiber to which they are applied, or results in the closure of the subject hair cuticle—thereby entrapping the discrete particle aggregates and/or agglomerates therein. In yet still another aspect of the present invention, one or more release methods may be applied to release the present discrete particle aggregates and/or agglomerates from the keratin fiber to which they are applied. These release methods may include deactivation using the application of light, application of temperature, change in pH, application of solvents, surface treatment, application of surfactants, application of dispersants and combinations thereof. Without wishing to be bound by theory, it is believed that application of the aforementioned release methods results in the breaking of chemical bonds created between the subject discrete particles aggregates and agglomerates and the subject keratin fibers, or by causing the subject hair cuticle to open—thereby releasing the particles, aggregates and/or agglomerates therefrom.

The compositions of the present invention may comprise from about 0.001% to about 15% by weight, preferably from about 0.01% to about 10% by weight, more preferably from about 0.1% to about 5% by weight of the discrete particle aggregates and/or agglomerates of the present invention.

Optional Ingredients and Adjuncts

Medium

The medium suitable for the application of the discrete particles may be selected from water, or a mixture of water and at least one organic solvent to dissolve the compounds that would not typically be sufficiently soluble in water, or may consist completely of a non-aqueous organic solvent. Suitable organic solvents for use herein include, but are not limited to: C1 to C4 lower alkanols (e.g., ethanol, propanol, isopropanol), aromatic alcohols (e.g. benzyl alcohol and phenoxyethanol); polyols and polyol ethers (e.g., carbitols, 2-butoxyethanol, propylene glycol, propylene glycol monomethyl ether, diethylene glycol monoethyl ether, monomethyl ether, hexylene glycol, glycerol, ethoxy glycol), and propylene carbonate. When present, organic solvents are typically present in an amount ranging from 1% to 30%, by weight, of the composition. Preferred solvents are water, ethanol, propanol, isopropanol, glycerol, 1,2-propylene glycol, hexylene glycol, ethoxy diglycol, and mixtures thereof.

In another aspect of the present invention, the discrete particle aggregate- and agglomerate-comprising compositions disclosed herein may comprise from about 1% to about 10%, by weight of the entire composition, of a non aqueous solvent; wherein said non-aqueous solvent is selected from the group consisting of: C1 to C4 lower alkanols (e.g., ethanol, propanol, isopropanol), aromatic alcohols (e.g. benzyl alcohol and phenoxyethanol); polyols and polyol ethers (e.g., carbitols, 2-butoxyethanol, propylene glycol, propylene glycol monomethyl ether, diethylene glycol monoethyl ether, monomethyl ether, hexylene glycol, glycerol, ethoxy glycol), and propylene carbonate.

The inventive compositions may, in some embodiments, further comprise additional optional components known, conventionally used, or otherwise effective for use in oxidative dye compositions, including but limited to: primary intermediate dye compounds; coupler dye compounds; direct dyes; anionic, cationic, nonionic, amphoteric or zwitterionic surfactants, or mixtures thereof; anionic, cationic, nonionic, amphoteric or zwitterionic polymers, or mixtures thereof; inorganic or organic thickeners; conditioning agents; oxidising agents; alkalising agents; antioxidants and radical scavengers; penetration agents; chelating and sequestering agents; fragrances; buffers; dispersing agents; peroxide stabilizing agents; natural ingredients, e.g. proteins and protein derivatives, and plant materials (e.g. aloe, chamomile and henna extracts); silicones (volatile or non-volatile, modified or non-modified), film-forming agents, ceramides, preserving agents; and opacifiers.

Some adjuvants referred to above, but not specifically described below, which are suitable are listed in the International Cosmetics Ingredient Dictionary and Handbook, (8^{th} ed.; The Cosmetics, Toiletry, and Fragrance Association). Particularly, vol. 2, sections 3 (Chemical Classes) and 4 (Functions) are useful in identifying specific adjuvants to achieve a particular purpose or multipurpose.

Oxidative Dye Compounds

The discrete particle aggregates and agglomerates disclosed herein may be present alone as dyeing agents, and can advantageously behave both like an oxidation base and like a coupler, e.g. self-coupling compounds. They may also be used in combination with one or more primary intermediates, and/or couplers, and in combination with one or more oxidizing agent. All known coupler and primary intermediate combinations are usable in the inventive compositions.

The compounds suitable for use in the inventive compositions (including those optionally added), in so far as they are bases, may be used as free bases or in the form of their physiologically compatible salts with organic or inorganic acids, such as hydrochloric, hydrobromic, citric, acetic, lactic, succinic, tartaric, or sulfuric acids, or, in so far as they have aromatic OH groups, in the form of their salts with bases, such as alkali phenolates.

Optional couplers, when present, are typically present in an amount such that in aggregate the concentration of couplers and the present discrete particle aggregates and/or agglomerates in the composition ranges from 0.002% to 10%, preferably from 0.01% to 5%, by weight, of the hair dyeing composition. Optional primary intermediates, when present, are present in an effective dyeing concentration, typically an amount from 0.001% to 10%, preferably from 0.01% to 5%, by weight, of the hair dyeing composition. The total amount of dye compounds (e.g., optional primary intermediates, optional coupler compounds, and the present discrete particle aggregates and/or agglomerates in the hair dyeing compositions of this invention will typically range from 0.002% to 20%, preferably from 0.04% to 10%, more preferably from 0.1% to 7%, by weight, of the hair dyeing composition.

These compounds are well known in the art, and include aromatic diamines, aminophenols, aromaticdiols and their derivatives (a representative but not exhaustive list of oxidation dye precursor can be found in Sagarin, “Cosmetic Science and Technology”, “Interscience, Special Edn. Vol. 2 pages 308 to 310). It is to be understood that the precursors detailed below are only by way of example and are not intended to limit the compositions and processes herein. These are:

1,7-Dihydroxynaphthalene (1,7-NAPHTHALENEDIOL), 1,3-Diaminobenzene (m-PHENYLENEDIAMINE), 1-Methyl-2,5-diaminobenzene (TOLUENE-2,5-DIAMINE), 1,4-Diaminobenzene (p-PHENYLENEDIAMINE), 1,3-Dihydroxybenzene (RESORCINOL), 1,3-Dihydroxy-4-chlorobenzene, (4-CHLORORESORCINOL), 1-Hydroxy-2-aminobenzene, (o-AMINOPHENOL), 1-Hydroxy-3-aminobenzene (m-AMINOPHENOL), 1-Hydroxy-4-amino-benzene (p-AMINOPHENOL), 1-Hydroxynaphthalene (1-NAPHTHOL), 1,5-Dihydroxynaphthalene (1,5-NAPHTHALENEDIOL), 2,7-dihydroxynaphthalene (2,7-NAPHTHELENEDIOL) 1-Hydroxy-2,4-diaminobenzene (4-DIAMINOPHENOL), 1,4-Dihydroxybenzene (HYDROQUINONE), 1-Hydroxy-4-methylaminobenzene (p-METHYLAMINOPHENOL), 6-Hydroxybenzo-morpholine (HYDROXYBENZOMORPHOLINE), 1-Methyl-2-hydroxy-4-aminobenzene (4-AMINO-2-HYDROXY-TOLUENE), 3,4-Diaminobenzoic acid (3,4-DIAMINOBENZOIC ACID), 1-Methyl-2-hydroxy-4-(2′-hydroxyethyl)aminobenzene (2-METHYL-5-HYDROXY-ETHYLAMINO-PHENOL), 1,2,4-Trihydroxybenzene (1,2,4-TRIHYDROXYBENZENE), 1-Phenol-3-methylpyrazol-5-on (PHENYLMETHYLPYRAZOLONE), 1-(2′-Hydroxyethyloxy)-2,4-diaminobenzene (2,4-DIAMINOPHENOXY-ETHANOL HCL), 1-Hydroxy-3-amino-2,4-dichlorobenzene (3-AMINO-2,4-DICHLORO-PHENOL), 1,3-Dihydroxy-2-methylbenzene (2-METHYLRESORCINOL), 1-Amino-4-bis-(2′-hydroxyethyl)aminobenzene (N,N-BIS(2-HYDROXY-ETHYL)-p-PHENYLENE-DIAMINE), 2,4,5,6-Tetraminopyrimidine (HC Red 16), 1-Hydroxy-3-methyl-4-aminobenzene (4-AMINO-m-CRESOL), 1-Hydroxy-2-amino-5-methylbenzene (6-AMINO-m-CRESOL), 1,3-Bis-(2,4-Diaminophenoxy)propane (1,3-BIS-(2,4-DIAMINO-PHENOXY)-PROPANE),1-(2′-Hydroxyethyl)-2,5-diaminobenzene (HYDROXYETHYL-p-PHENYLENE DIAMINE SULPHATE), 1-Methoxy-2-amino-4-(2′-hydroxyethylamino)benzene, (2-AMINO-4-HYDROXYETHYLAMINOANISOLE) 1-Hydroxy-2-methyl-5-amino-6-chlorobenzene (5-AMINO-6-CHLORO-o-CRESOL), 1-Hydroxy-2-amino-6-methylbenzene (6-AMINO-o-CRESOL), 1-(2′-Hydroxyethyl)-amino-3,4-methylenedioxybenzene (HYDROXYETHYL-3,4-METHYLENEDIOXY-ANILINE HCl), 2,6-Dihydroxy-3,4-dimethylpyridine (2,6-DIHYDROXY-3,4-DIMETHYLPYRIDINE), 3,5-Diamino-2,6-dimethoxypyridine (2,6-DIMETHOXY-3,5-PYRIDINEDIAMINE), 5,6-Dihydroxyindole (,DIHYDROXY-INDOLE), 4-Amino-2-aminomethylphenol (2-AMINOETHYL-p-AMINO-PHENOL HCl), 2,4-Diamino-5-methylphenetol (2,4-DIAMINO-5-METHYL-PHENETOLE HCl), 2,4-Diamino-5-(2′-hydroxyethyloxy)toluene (2,4-DIAMINO-5-METHYLPHENOXYETHANOL HCl), 5-Amino-4-chloro-2-methylphenol (5-AMINO-4-CHLORO-o-CRESOL), 4-Amino-1-hydroxy-2-(2′-hydroxyethylaminomethyl)benzene HYDROXYETHYLAMINOMETHYL-p-AMINO PHENOL HCl), 4-Amino-1-hydroxy-2-methoxymethylbenzene (2-METHOXYMETHYL-p-AMINOPHENOL HCl), 1,3-Bis(N(2-Hydroxyethyl)N(4-amino-phenyl)amino)-2-propanol (HYDROXYPROPYL-BIS-(N-HYDROXY-ETHYL-p-PHENYLENEDIAMINE)HCL), 6-Hydorxyindole (6-HYDROXY-INDOLE), 2,3-Indolinedione (ISATIN), 3-Amino-2-methylamino-6-methoxypyridine (HC BLUE NO. 7), 1-Phenyl-3-methyl-5-pyrazolone-2,4-dihydro-5,2-phenyl-3H-pyrazole-3-one, 2-Amino-3-hydroxypyridine (2-AMINO-3-HYDROXYPYRIDINE), 5-Amino-salicylic acid, 1-Methyl-2,6-bis(2-hydroxy-ethylamino)benzene (2,6-HYDROXYETHYLAMINO-TOLUENE), 4-Hydroxy-2,5,6-triaminopyrimidine (2,5,6-TRIAMINO-4-PYRIMIDINOL SULPHATE), 2,2′-[1,2-Ethanediyl-bis-(oxy-2,1-ethanediyloxy)]-bis-benzene-1,4-diamine (PEG-3,2′,2′-DI-p-PHENYLENEDIAMINE), 5,6-Dihydroxyindoline (DIHYDROXYINDOLINE), N,N-Dimethyl-3-ureidoaniline (m-DIMETHYL-AMINO-PHENYLUREA), 2,4-Diamino-5-fluortoluenesulfatehydrate (4-FLUORO-6-METHYL-m-PHENYLENEDIAMINE SULPHATE) and 1-Acetoxy-2-methylnaphthalene (1-HYDROXYYETHYL-4,5-DIAMINOPYRAZOLE SULPHATE). These can be used in the molecular form or in the form of peroxide-compatible salts.

The hair colouring compositions of the present invention may also include non oxidative hair dyes. i.e. direct dyes which may be used alone or in combination with the above described oxidative dyes. Suitable direct dyes include azo or anthraquinone dyes and nitro derivatives of the benzene series and or melanin precursors and mixtures thereof. Such direct dyes are particularly useful to deliver shade modification or highlights. Particularly preferred are Basic Red 51, Basic Orange 31, Basic Yellow 87 and mixtures thereof.

Oxidizing Agent

The developer compositions suitable for use with the inventive compositions may comprise an oxidizing agent, present in an amount sufficient to bleach melanin pigment in hair and/or cause formation of dye chromophores from oxidative dye precursors (including primary intermediates and/or couplers when present). Typically, such an amount ranges from 1% to 20%, preferably from 3% to 15%, more preferably from 6% to 12%, by weight, of the developer composition. Inorganic peroxygen materials capable of yielding hydrogen peroxide in an aqueous medium are preferred, and include but are not limited to: hydrogen peroxide; inorganic alkali metal peroxides (e.g. sodium periodate and sodium peroxide); organic peroxides (e.g. urea peroxide, melamine peroxide); inorganic perhydrate salt bleaching compounds (e.g. alkali metal salts of perborates, percarbonates, perphosphates, persilicates, and persulphates, preferably sodium salts thereof), which may be incorporated as monohydrates, tetrahydrates, etc.; alkali metal bromates; enzymes; and mixtures thereof. Preferred is hydrogen peroxide. In another aspect of the present invention, the discrete particle-, aggregate- and/or agglomerate-comprising compositions of the present invention may further comprise from about 1% to about 10%, by weight of the entire composition, of a bleaching agent selected from the group consisting of: peroxygen oxidizing agents, especially alkaline hydrogen peroxide, persulfate salts, peroxyacids or mixtures thereof inorganic alkali metal peroxides such as sodium periodate and sodium peroxide and organic peroxides such as urea peroxide, melamine peroxide, and inorganic perhydrate salt bleaching compounds, such as the alkali metal salts of perborates, percarbonates, perphosphates, persilicates, persulphates and combinations thereof.

Thickeners

The inventive compositions may comprise a thickener in an amount sufficient to provide the composition with a viscosity so that it can be readily applied to the hair without unduly dripping off the hair and causing mess. Typically, such an amount will be at least 0.1%, preferably at least 0.5%, more preferably, at least 1%, by weight, of the hair composition.

Preferred for use herein are salt tolerant thickeners, including but not limited to: xanthan, guar, hydroxypropyl guar, scleroglucan, methyl cellulose, ethyl cellulose (available as AQUACOTE™), hydroxyethyl cellulose (NATROSOL™), carboxymethyl cellulose, hydroxypropylmethyl cellulose, microcrystalline cellulose, hydroxybutylmethyl cellulose, hydroxypropyl cellulose (available as KLUCEL™), hydroxyethyl ethyl cellulose, cetyl hydroxyethyl cellulose (available as NATROSOL™ Plus 330), N-vinylpyrollidone (available as POVIDONE™), Acrylates/Ceteth-20 Itaconate Copolymer (available as STRUCTURE™ 3001), hydroxypropyl starch phosphate (available as STRUCTURE™ ZEA), polyethoxylated urethanes or polycarbamyl polyglycol ester (e.g. PEG-150/Decyl/SMDI copolymer (available as ACULYN™ 44), PEG-150/Stearyl/SMDI copolymer available as ACULYN™ 46), trihydroxystearin (available as THIXCIN™), acrylates copolymer (e.g. available as ACULYN™ 33) or hydrophobically modified acrylate copolymers (e.g. Acrylates/Steareth-20 Methacrylate Copolymer (available as ACULYN™ 22), non-ionic amphophilic polymers comprising at least one fatty chain and at least one hydrophilic unit selected from polyether urethanes comprising at least one fatty chain, and blends of Ceteth—10 phosphate, Di-cetyl phosphate and Cetearyl alcohol (available as CRODAFOS™ CES).

Swelling Agents

The inventive compositions may further comprise from about 1% to about 10%, by weight of the entire composition, of a swelling agent for swelling the porous material to which they are applied, wherein said swelling agent is selected from the group consisting of: ammonia, monoethanolamine, propanolamine, ammonium carbonate, metal salts of carbonates, silicates and their salts, ammonium hydroxide and mixtures thereof.

Dispersing Agents

The inventive compositions may further comprise from about 1% to about 10%, by weight of the entire composition, of a dispersing agent to prevent unintended aggregation; wherein said dispersing agent is selected from the group consisting of: cellulose, aniline, anionic polyelectrolytes (e.g. polyacrylic acids and its salt), copolymers of acrylic acid, maleic acid anhydride, sulfonic acids with acrylic-, methacrylic- and vinylic monomers, low molecular weight surfactants (nonylphenol ethoxylates) and high molecular weight systems.

Chelants

The inventive compositions may comprise chelants in an amount sufficient to reduce the amount of metals available to interact with formulation components, particularly oxidizing agents, more particularly peroxides. Typically such an amount will range from at least 0.25%, preferably at least 0.5%, by weight, of the composition. Suitable chelants for use herein include but are not limited to: diamine-N,N′-dipolyacid, monoamine monoamide-N,N′-dipolyacid, and N,N′-bis(2-hydroxybenzyl)ethylenediamine-N,N′-diacetic acid chelants (preferably EDDS (ethylenediaminedisuccinic acid)), carboxylic acids (preferably aminocarboxylic acids), phosphonic acids (preferably aminophosphonic acids) and polyphosphoric acids (in particular straight polyphosphoric acids), their salts and derivatives.

pH Modifiers and Buffering Agents

The inventive compositions may further comprise a pH modifier and/or buffering agent in an amount that is sufficiently effective to adjust the pH of the composition to fall within a range from 3 to 13, preferably from 8 to 12, more preferably from 9 to 11. Suitable pH modifiers and/or buffering agents for use herein include, but are not limited to: ammonia, alkanolamides such as monoethanolamine, diethanolamine, triethanolamine, monopropanolamine, dipropanolamine, tripropanolamine, tripropanolamine, 2-amino-2-methyl-1-propanol, and 2-amino-2-hydroxymethyl-1,3,-propandiol and guanidium salts, alkali metal and ammonium hydroxides and carbonates, preferably sodium hydroxide and ammonium carbonate, and acidulents such as inorganic and inorganic acids, e.g., phosphoric acid, acetic acid, ascorbic acid, citric acid or tartaric acid, hydrochloric acid, and mixtures thereof. In another aspect of the present invention, the discrete particle-, aggregate- and agglomerate-comprising compositions of the present invention may further comprise from about 1% to about 10%, by weight of the entire composition, of a pH modifier and/or buffering agent as described hereinbefore.

Surface-Modifying Agents

The inventive compositions may further be treated with a surface modified coating that changes it physical and/or chemical characteristics. Said surface modifying agent may be selected from the group consisting of: cellulose, aniline, anionic polyelectrolytes (e.g. polyacrylic acids and its salt), copolymers of acrylic acid, maleic acid anhydride, sulfonic acids with acrylic-, methacrylic- and vinylic monomers, low molecular weight surfactants (nonylphenol ethoxylates) and high molecular weight systems.

Carbonate Ion Source and Radical Scavenger System—

The inventive compositions may further comprise a system comprising the combination of at least one source of peroxymonocarbonate ions, preferably formed insitu from a source of hydrogen peroxide and a carbonate ion source, at least one source of alkalizing agents and preferably e source of radical scavenger, (as defined hereinafter), in an amount to sufficiently reduce odour and the damage to the hair fibers. Typically, such an amount will range from 0.1% to 15%, preferably 0.1% to 10%, more preferably 1% to 8%, by weight of the hair dyeing composition and/or the hair dyeing product composition, of the carbonate ion, and from 0.1% to 10%, preferably from 1% to 7%, by weight of the composition, of radical scavenger and from 0.1 to 10%, preferably from 0.5 to 5% of the alkalising agent. Preferably, the radical scavenger is present at an amount such that the ratio of radical scavenger to carbonate ion is from 1:1 to 1:4. The radical scavenger is preferably selected such that it is not an identical species as the alkalizing agent.

According to the present invention the compositions thus may also comprise at least a source of carbonate ions or carbamate ions or hydrocarbonate ions or any mixture thereof. Any source of these ions may be utilized. Suitable sources for use herein include sodium, potassium, guanidine, arginine, lithium, calcium, magnesium, barium, ammonium salts of carbonate, carbamate and hydrocarbonate ions and mixtures thereof such as sodium carbonate, sodium hydrogen carbonate, potassium carbonate, potassium hydrogen carbonate, guanidine carbonate, guanidine hydrogen carbonate, lithium carbonate, calcium carbonate, magnesium carbonate, barium carbonate, ammonium carbonate, ammonium hydrogen carbonate and mixtures thereof. Percarbonate salts may also be utilized to provide both the source of carbonate ions and oxidizing agent. Preferred sources of carbonate ions, carbamate and hydrocarbonate ions are sodium hydrogen carbonate, potassium hydrogen carbonate, ammonium carbamate and mixtures thereof.

According to the present invention the composition may also thus comprise at least one source of alkalizing agent, preferably a source of ammonium ions and or ammonia. Any agent known in the art may be used such as alkanolamides for example monoethanolamine, diethanolamine, triethanolamine, monopropanolamine, dipropanolamine, tripropanolamine, 2-amino-2-methyl-1,3-propanediol, 2-amino-2-methyl-1-propanol, and 2-amino-2-hydroxymethyl-1,3-propanediol and guanidium salts. Particularly, preferred alkalizing agents are those which provide a source of ammonium ions. Any source of ammonium ions is suitable for use herein. Preferred sources include ammonium chloride, ammonium sulphate, ammonium nitrate, ammonium phosphate, ammonium acetate, ammonium carbonate, ammonium hydrogen carbonate, ammonium carbamate, ammonium hydroxide, percarbonate salts, ammonia and mixtures thereof. Particularly preferred are ammonium carbonate, ammonium carbamate, ammonia and mixtures thereof.

The radical scavenger is a species that can react with a carbonate radical to convert the carbonate radical by a series of fast reactions to a less reactive species. Suitable radical scavengers for use herein include compounds according to the general formula:
R¹—Y—C(H)(R³)—R⁴—(C(H)(R⁵)—Y—R⁶)ₙ (I)

wherein Y is NR², O, or S, preferably NR², n is 0 to 2, and wherein R⁴ is monovalent or divalent and is selected from: (a) substituted or unsubstituted, straight or branched, alkyl, mono- or poly-unsaturated alkyl, heteroalkyl, aliphatic, heteroaliphatic, or heteroolefinic systems, (b) substituted or unsubstituted, mono- or poly-cyclic aliphatic, aryl, or heterocyclic systems, or (c) substituted or unsubstituted, mono-, poly-, or per-fluoro alkyl systems; the systems of (a), (b) and (c) comprising from 1 to 12 carbon atoms and 0 to 5 heteroatoms selected from O, S, N, P, and Si; and wherein R⁴ can be connected to R³ or R⁵ to create a 5, 6 or 7 membered ring; and wherein R¹, R², R³, R⁵, and R⁶ are monovalent and are selected independently from: (a), (b) and (c) described herein above, or H.

Preferably, R⁴ is selected from: (a) substituted or unsubstituted, straight or branched, alkyl, heteroalkyl, aliphatic, heteroaliphatic, or heteroolefinic systems, (b) substituted or unsubstituted, mono- or poly-cyclic aliphatic, aryl, or heterocyclic systems, or (c) substituted or unsubstituted, mono-, poly-, or per-fluoro alkyl systems; more preferably R⁴ is selected from (a) substituted or unsubstituted, straight or branched, alkyl, heteroalkyl, aliphatic, or heteroaliphatic systems, (b) substituted or unsubstituted, aryl, or heterocyclic systems, or (c) substituted or unsubstituted, mono-, poly-, or per-fluoro alkyl systems; more preferably substituted or unsubstituted, straight or branched, alkyl, or heteroalkyl systems.

Preferably, the R⁴ systems of (a), (b), and (c), described herein above, comprise from 1 to 8 carbon atoms, preferably from 1 to 6, more preferably from 1 to 4 carbon atoms and from 0 to 3 heteroatoms; preferably from 0 to 2 heteroatoms; most preferably from 0 to 1 heteroatoms. Where the systems contain heteroatoms, preferably they contain 1 heteroatom. Preferred heteroatoms include O, S, and N; more preferred are O, and N; and most preferred is O.

Preferably, R¹, R², R³, R⁵, and R⁶ are selected independently from any of the systems defined for R⁴ above, and H.

In alternative embodiments, any of R¹, R², R³, R⁴, R⁵, and R⁶ groups are substituted. Preferably, the substituent(s) is selected from: (a) the group of C-linked monovalent substituents consisting of: (i) substituted or unsubstituted, straight or branched, alkyl, mono- or poly-unsaturated alkyl, heteroalkyl, aliphatic, heteroaliphatic, or heteroolefinic systems, (ii) substituted or unsubstituted, mono- or poly-cyclic aliphatic, aryl, or heterocyclic systems, or (iii) substituted or unsubstituted, mono-, poly-, or per-fluoro alkyl systems; said systems of (i), (ii) and (iii) comprising from 1 to 10 carbon atoms and 0 to 5 heteroatoms selected from O, S, N, P, and Si; (b) the group of S-linked monovalent substituents consisting of SA¹, SCN, SO₂A¹, SO₃A¹, SSA¹, SOA¹, SO₂NA¹A², SNA¹A², and SONA¹A²; (c) the group of O-linked monovalent substituents consisting of OA¹, OCN and ONA¹A²; (d) the group of N-linked monovalent substituents consisting of NA¹A², (NA¹A²A³)⁺, NC, NA¹OA², NA¹SA², NCO, NCS, NO₂, N═NA¹, N═NOA¹, NA¹CN, NA¹NA²A³; (e) the group of monovalent substituents consisting of COOA¹, CON₃, CONA¹₂, CONA¹COA², C(═NA¹)NA¹A², CHO, CHS, CN, NC, and X; and (f) the group consisting fluoroalkyl monovalent substituents consisting of mono-, poly-, or per-fluoro alkyl systems comprising from 1 to 12 carbon atoms and 0 to 4 heteroatoms.

For the groups (b) to (e), described above, A¹, A², and A³ are monovalent and are independently selected from: (1) H, (2) substituted or unsubstituted, straight or branched, alkyl, mono- or poly-unsaturated alkyl, heteroalkyl, aliphatic, heteroaliphatic, or heteroolefinic systems, (3) substituted or unsubstituted, mono- or poly-cyclic aliphatic, aryl, or heterocyclic systems, or (4) substituted or unsubstituted, mono-, poly-, or per-fluoro alkyl systems; said systems of (2), (3) and (4) comprising from 1 to 10 carbon atoms and 0 to 5 heteroatoms selected from O, S, N, P, and Si; and wherein X is a halogen selected from the group consisting of F, Cl, Br, and I.

Preferred radical scavengers according to the present invention are selected from the classes of alkanolamines, amino sugars, amino acids, esters of amino acids and mixtures thereof. Particularly preferred compounds are: monoethanolamine, 3-amino-1-propanol, 4-amino-1-butanol, 5-amino-1-pentanol, 1-amino-2-propanol, 1-amino-2-butanol, 1-amino-2-pentanol, 1-amino-3-pentanol, 1-amino-4-pentanol, 3-amino-2-methylpropan-1-ol, 1-amino-2-methylpropan-2-ol, 3-aminopropane-1,2-diol, glucosamine, N-acetylglucosamine, glycine, arginine, lysine, proline, glutamine, histidine, sarcosine, serine, glutamic acid, tryptophan, and mixtures thereof, and the salts such as the potassium, sodium and ammonium salts thereof and mixtures thereof.

Especially preferred compounds are glycine, sarcosine, lysine, serine, 2 methoxyethylamine, glucosamine, glutamic acid, morpholine, piperdine, ethylamine, 3 amino-1-propanol and mixtures thereof.

Conditioning Agents

The compositions of the present invention may comprise or are used in combination with a composition comprising a conditioning agent. Conditioning agents suitable for use herein are selected from silicone materials, amino silicones, fatty alcohols, polymeric resins, polyol carboxylic acid esters, cationic polymers, cationic surfactants, insoluble oils and oil derived materials and mixtures thereof. Additional materials include mineral oils and other oils such as glycerin and sorbitol.

The conditioning agent will generally be used at levels of from about 0.05% to about 20% by weight of the hair dyeing product composition, preferably of from about 0.1% to about 15%, more preferably of from about 0.2% to about 10%, even more preferably of from about 0.2% to about 2%.

Particularly useful conditioning materials are cationic polymers. Conditioners of cationic polymer type may be chosen from those already known by those skilled in the art as improving at least one cosmetic properties of keratin fibers treated with a cosmetic composition. Cationic polymers can be chosen from those comprising units of at least one amine group chosen from primary, secondary, tertiary and quaternary amine groups that may either form part of the main polymer chain, or be borne by a side substituent that is directly attached to the main polymer chain. Such cationic polymers generally have a number average molecular mass ranging from 500 to 5×10⁶, or more preferably from 1000 to 3×10⁶.

Adjunct Ingredients

The inventive compositions may further comprise one or more adjunct ingredients selected from the group consisting of: conditioning agents, penetration enhancers, polymer attachment vehicles, surface modifiers, retention enhancers, release promoters, coloring agents, agglomeration modifiers, fragrances and combinations thereof.

Methods of Manufacture

The compositions of this invention may be manufactured using conventional methods. The hair compositions are preferably provided as a cream, gel, mousse or spray. The hair dyeing compositions may be formed as solutions, preferably as aqueous or aqueous-alcohol solutions. The hair dye product compositions may preferably be formed as thick liquids, creams, gels, or emulsions whose composition is a mixture of the dye compound and other dye ingredients with conventional cosmetic additive ingredients suitable for the particular preparation and preferably with the developer composition.

Methods of Use

The compositions of the present invention are applied either directly onto the hair or via a device such as a comb or cloth. The compositions may also be used in combination with a hair dye or hair bleach product composition. In such embodiments the discrete particles can be added directly into the hair dye or bleach composition, either to the developer composition or the composition containing the alkaliser and/or dye precursors. In addition, the discrete particle compostion can be added to the hair directly before the hair dye or bleaching composition is applied or directly after the hair dye or bleaching composition is applied. Alternatively the discrete particle compoostion can be applied any time between the hair colouring events, for example if containing coloured particles the formulation can be applied to replace the colour lost from the disappearance of the oxidative dyes from wash/light induced fade.

Together, the hair dyeing composition and the developer composition form a system for dyeing hair. This system may be provided as a kit comprising in a single package separate containers of the hair dyeing composition, the developer composition, the optional conditioner or other hair treatment product, and instructions for use.

### Discrete Particle Size Measurement Methods

1) Milling of samples

A 5% wt/wt solution of discrete particles is made with distilled water. The solution is mixed with a spoon until all the discrete particles are wetted and are suspended in the water. The 5% solution is then diluted 10-fold with distilled water. The pH of this solution is then adjusted using either ammonium hydroxide (30% active) or acetic acid to the required pH. The solution is then placed in an Ultra Turrax mill (model T-25 basic SI, manufactured by IKA Works). A drill bit is fitted that can mill samples to a 3 μm particle size. The mill is mixed at 6500 rpm for 1 minute and then the speed gradually increased to 24,000 rpm for a further 9 minutes.

2) Sonication of Samples

The solution of discrete particles in distilled water at the required pH is placed in the sonicator (Misonix Model 3000 fitted with a standard probe utilizing a half inch horn). The sonication programme is set for a run time of 3 minutes with a pulse on for 1.5 secs and a pulse off for 1.5 secs. The total run time is 6 minutes. The power level is set to 6.5 which is an equivalent of 48-51 watts of energy.

3) Particle Size Measurement Using the Horiba

The particle size in a diluted solution is measured using a Horiba Model LA-930, Laser Scattering, Particle Size and Distribution Analyser.

The Horiba reservoir is filled with distilled water and mixed at a speed of 4. The refractive index on the instrument is set to match the refractive index of the discrete particles to be measured. The lenses of the instrument are then aligned. A 5% wt/wt solution of the discrete particles is then diluted 10-fold with distilled water and pH adjusted to the desired pH using either ammonium hydroxide (30% active) or acetic acid. This solution is then added into the reservoir until the % T reads from 95% to 97%. The particle size distribution is then measured.

4) Particle Size Measurement Using the Malvern

The particle size in the hair compositions is measured using a Malvern Zetano Sizer S instrument. This instrument is a backscattering dynamic light scattering instrument that uses a very high angle of scattering detection through the use of fibre optics. This high angle allows for the detection of very small particles.

The hair composition is loaded into standard disposable spectroscopic 10 mm cells. The sides of the cell are cleaned with methanol and lens paper. The cell is then capped and inserted into the instrument cell container. The particle size is then measured using the standard measurement programme giving an output of the particle size distribution.

### EXAMPLES

### Examples of Formation of Discrete Particle Aggregates and/or Agglomerates

Discrete particle aggregates and/or agglomerates can be prepared and employed to achieve color effects (and other physical and/or chemical characteristics) individually and in the absence of oxidative and/or direct dyes, by adding an insoluble solid powder of a metal oxide (or other dyeing agent) to an aqueous or non aqueous solution, or a mixture of the two.

### Example 1

5 g of iron oxide (supplied by Nanotechnologies) was added to 95 g of deionized water and mixed with a spoon until all the discrete particles were wetted and suspended. The starting mean particle size was 6.5 μm. The pH of the mixture was then adjusted to pH 9.4 with acetic acid. The mixture was then milled for one minute at 6500 rpm using an Ultra Turrax mill with a mill bit designed to mill samples to 3 micron size. After one minute the mixing speed was increased to 24,000 rpm for a total run time of 9 minutes. After milling the mean particle size was 1.59 μm. The mixture was then sonicated using a Misonix Model 3000 sonicator with a standard probe utilising a half inch horn. The particles were exposed to 51 watts of energy for a total of 6 minutes. After sonication the particle size and distribution was measured using a Horiba Model LA-930 Laser scattering, particle size and distribution analyser according to the manufacturer instructions. The final mean particle size was 185 nm

### Example 2

5 g of titanium dioxide (supplied by Nanophase) was added to 95 g of deionized water and mixed with a spoon until all the discrete particles were wetted and suspended. The starting particle size was 0.36 μm. The mixture was then sonicated using a Misonix Model 3000 sonicator with a standard probe utilising a half inch horn. The particles were exposed to 51 watts of energy for a total of 6 minutes. After sonication the particle size and distribution was measured using a Horiba Model LA-930 Laser scattering, particle size and distribution analyser according to the manufacturer instructions. The final particle size was 290 nm

### Example 3

5 g of silicon dioxide (supplied by Nanophase) was added to 95 g of deionized water and mixed with a spoon until all the discrete particles were wetted and suspended. The starting particle size was 99 nm. No milling or sonication was required for this sample.

The particles of the correct size were added to the hair composition or the hair dyeing or bleaching compositions as illustrated below. Before application to the hair the hair compositions were checked to confirm that the particle size distribution was still as seen in solution. This was done using the Malvern Zetanano Sizer S instrument.

After application to the hair, scanning electron microscopy was used to confirm that the location of the discrete particle was within the cuticle structure of the hair.

(1) Shampoo Formulation

| | % Weight |
|---|---|
| Sodium laureth sulphate | 12.0 |
| Cocamidopropyl hetaine | 3.0 |
| Disodium laureth sulfosuccinate | 3.0 |
| Gluadin ® WQ | 1.2 |
| Diethylene glycol monolauryl ether | 3.0 |
| Propylene glycol | 1.0 |
| Iron Oxide Particles | 0.5 |
| Zinc Oxide Particles | 3.0 |
| Carbon Black Particles | 5.0 |
| Water | q.s. to 100 |

(2)(a) Shampoo/conditioner Formulation

| Supplier name/Description | 1 | 2 | 3 |
|---|---|---|---|
| Water-USP Purified & Minors | Q.S. to | Q.S. to | Q.S. to |
| | 100 | 100 | 100 |
| Ammonium Laureth Sulfate | 10.0000 | 12.5000 | 12.0000 |
| Ammonium Lauryl Sulfate | 6.0000 | 1.5000 | 2.0000 |
| Cocamidopropyl Betaine | | 2.7000 | |
| Sodium Lauroamphoacetate | | | 2.0000 |
| Cocamide MEA | 0.8000 | 0.8000 | 0.8000 |
| Cetyl Alcohol | 0.9000 | 0.6000 | 0.6000 |
| Ethylene Glycol Distearate | 1.5000 | 1.5000 | |
| Dimethicone Viscasil 330,000 | 1.3500 | | |
| Dow Corning 1664 300 nm/60 M emulsion | | 1.0000 | |
| Polyquaternium-10 (LR30M) | 0.5000 | 0.1500 | 0.5000 |
| Polyox PEG7M | 0.1000 | | |
| Puresyn 6 (1-decene homopolymer) | 0.3000 | | |
| Perfume | 0.5000 | 0.5000 | 0.5000 |
| Citric Acid | 0.0400 | 0.0400 | 0.4000 |
| Sodium Citrate Dihydrate | 0.3972 | 0.3972 | 0.3972 |
| Disodium EDTA | 0.0993 | 0.0993 | 0.0993 |
| Kathon | 0.0005 | 0.0005 | 0.0005 |
| Sodium Benzoate | 0.2500 | 0.2500 | 0.2500 |
| Sodium Chloride | 0-3 | 0-3 | 0-3 |
| Ammonium Xylene Sulfonate | 0-3 | 0-3 | 0-3 |
| Iron oxide partciels | 0.5 | 0.5 | 0.5 |
| Zinc oxide particles | 3.0 | 3.0 | 3.0 |
| Carbon black particles | 5.0 | 5.0 | 5.0 |

(2)(b) Hair conditioner formulation

| Components | Example 1 | Example 2 | Example 3 |
|---|---|---|---|
| Cetyl Alcohol *1 | 2.0 | 2.5 | 2.0 |
| Stearyl Alcohol *2 | 3.6 | 4.5 | 3.6 |
| Stearamidopropyl Dimethylamine *3 | 1.6 | 2.0 | 1.6 |
| l-Glutamic acid *4 | 0.512 | 0.64 | 0.512 |
| Zinc pyrithione *5 | 2.0 | 2.0 | 2.0 |
| Benzyl alcohol | 0.4 | 0.4 | 0.4 |
| Phenoxy Ethanol | 0.3 | 0.3 | 0.3 |
| Methyl Paraben | 0.2 | 0.2 | 0.2 |
| Propyl Paraben | 0.1 | 0.1 | 0.1 |
| Silicone Blend *6 | 3.36 | 4.37 | 3.36 |
| Natural Menthol *19 | — | — | 0.4 |
| Perfume | 0.4 | 0.4 | 0.4 |
| 3-pyridinecarboxy acid amide | 0.05 | 0.05 | 0.05 |
| dl-Alpha tocopherol acetate | 0.05 | 0.05 | 0.05 |
| Hydrolyzed collagen *7 | 0.01 | 0.01 | 0.01 |
| Panthenol *8 | 0.05 | 0.05 | 0.05 |
| Panthenyl Ethyl Ether *9 | 0.05 | 0.05 | 0.05 |
| Octyl methoxycinnamate | 0.09 | 0.09 | 0.09 |
| Benzophenone-3 | 0.09 | 0.09 | 0.09 |
| Citric acid - (adjust to pH 3-7) | | | |
| Iron Oxide particles | 0.5 | 0.5 | 0.5 |
| Zinc oxide particles | 3.0 | 3.0 | 3.0 |
| Carbon black particles | 5.0 | 5.0 | 5.0 |
| Deionized water | q.s 100% | q.s. 100% | q.s 100% |

Compositions

| Components | Example 4 | Example 5 | Example 6 |
|---|---|---|---|
| Cetyl Alcohol *1 | 2.6 | 2.0 | 2.6 |
| Stearyl Alcohol *2 | 4.6 | 3.6 | 4.6 |
| Stearamidopropyl Dimethylamine *3 | 1.8 | 1.6 | 1.8 |
| l-Glutamic acid *4 | 0.6 | 0.5 | 0.6 |
| Pentaerythritol Tetraisostearate *11 | 1.0 | 0.5 | 1.0 |
| Polypropylene Glycol *18 | 4.5 | 4.0 | 4.5 |
| Zinc pyrithione *5 | 2.0 | 2.0 | 2.0 |
| Benzyl alcohol | 0.4 | 0.4 | 0.4 |
| Phenoxy Ethanol | 0.3 | 0.3 | 0.3 |
| Methyl Paraben | 0.2 | 0.2 | 0.2 |
| Propyl Paraben | 0.1 | 0.1 | 0.1 |
| Natural Menthol *19 | — | — | 0.4 |
| Perfume | 0.4 | 0.4 | 0.4 |
| 3-pyridinecarboxy acid amide | 0.05 | 0.05 | 0.05 |
| dl-Alpha tocopherol acetate | 0.05 | 0.05 | 0.05 |
| Hydrolyzed collagen *7 | 0.01 | 0.01 | 0.01 |
| Panthenol *8 | 0.05 | 0.05 | 0.05 |
| Panthenyl Ethyl Ether *9 | 0.05 | 0.05 | 0.05 |
| Octyl methoxycinnamate | 0.09 | 0.09 | 0.09 |
| Benzophenone-3 | 0.09 | 0.09 | 0.09 |
| Citric Acid | amount necessary to adjust pH 3-7 | | |
| Iron Oxide particles | 0.5 | 0.5 | 0.5 |
| Zinc oxide particles | 3.0 | 3.0 | 3.0 |
| Carbon black particles | 5.0 | 5.0 | 5.0 |

| | |
|---|---|
| Deionized Water | q.s. to 100% |

*1 Cetyl Alcohol: Konol series available from Shin Nihon Rika.
*2 Stearyl Alcohol: Konol series available from Shin Nihon Rika.
*3 Stearamidopropyl Dimethylamine: SAPDMA available from Inolex.
*4 l-Glutamic acid: l-Glutamic acid (cosmetic grade) available from Ajinomoto.
*5 Zinc pyrithinone: Zinc pyrithione U/2 available from Olin
*6 Silicone Blend: SE 76 available from General Electric
*7 Hydrolyzed collagen: Peptein 2000 available from Hormel.
*8 Panthenol: available from Roche.
*9 Panthenyl Ethyl Ether: available from Roche.
*11 Pentaerythritol Tetraisostearate: KAK PTI obtained by Kokyu alcohol.
*18 Polypropylene Glycol: PP2000 available from Sanyo Kasei.
*19 Natural Menthol: Menthol Crystal available from Dr Kolb.

(3) Hair Colour Formulation

The discrete particles or combination of particles can be added to either the hair dye composition that contains the alkaliser and the dye precursors or the developer composition. The developer and dye composition is mixed in a 1:1 ratio before application to hair. The product is left on the hair for 30 mins and then rinsed off.

(i) Hair dye Composition

| | Composition Example Number | | | | | | | | | |
|---|---|---|---|---|---|---|---|---|---|---|
| Ingredient | 4 | 5 | 6 | 7 | 8 | 9 | 10 | 11 | 12 | 13 |
| Sodium Sulphite | — | — | 0.1 | 0.1 | 0.1 | 0.1 | 0.1 | 0.3 | 0.1 | — |
| Ascorbic Acid | 0.5 | 0.1 | — | 0.1 | 0.3 | — | 0.6 | 0.1 | 0.1 | 0.2 |
| Ammonium Hydroxide | 6 | 8 | 8 | 7 | 8 | 9 | 10 | 8 | 8 | 10 |
| Ethylenediamedisuccinic acid | — | — | — | 1 | — | 1 | — | 0.5 | — | 1.5 |
| Oleth 5 | 1 | 2 | 3 | 0.5 | 1 | 1.5 | — | 0.8 | 2 | 1 |
| Oleth 2 | 0.8 | — | 0.8 | 0.8 | 1.5 | 2 | 0.8 | 0.5 | 0.8 | 2.5 |
| Oleic Acid | 0.9 | 1 | — | 0.3 | — | 0.9 | 0.9 | 0.8 | 1.1 | 0.9 |
| Soytrimonium chloride | 7 | 6 | 6 | 7 | 7 | — | — | 8 | 5 | 7 |
| Cocamide DEA | 3 | 1 | 1 | 3 | 0.5 | 0.8 | — | — | 3 | 2 |
| EDTA (Na₄ salt) | 0.1 | 0.1 | 0.1 | 0.1 | 0.1 | — | 0.1 | 0.1 | 0.1 | 0.1 |
| 1,4 diaminobenzene | 0.8 | 0.5 | — | 0.5 | 0.8 | — | 0.5 | 0.6 | 0.5 | 0.8 |
| 4-aminophenol | 0.2 | — | — | 0.1 | 0.2 | — | — | 0.2 | 0.1 | 0.2 |
| 3-aminophenol | 0.5 | 0.5 | — | 0.6 | 1 | — | 0.5 | 1 | 0.6 | 1 |
| 4-amino-3-methylphenol | 0.2 | — | — | 0.2 | 0.2 | — | 1 | | 0.2 | 0.3 |
| 2-(4,5-diamino-1H-pyrazol-1-yl)ethanol | — | 0.5 | — | — | 0.5 | — | 0.5 | 1 | — | 0.3 |
| N,N-Bis(2-hydroxyethyl)-p- | — | 0.4 | — | 1 | 0.2 | — | 0.2 | — | 0.2 | 0.3 |
| phenylenediamine | | | | | | | | | | |
| 2-aminophenol | 1 | 1 | — | — | — | — | — | — | — | — |
| 3,5-dimethyl-2-aminophenol | — | — | — | 1 | — | — | — | — | — | — |
| 3,5-dimethoxymethyl 2- | 1 | 0.5 | — | — | 1 | 1.5 | 1 | — | — | — |
| aminophenol | | | | | | | | | | |
| 3,5-diethyl 2-aminophenol | — | — | 1 | 0.8 | — | — | — | 1 | 1 | 1 |
| Propylene Glycol | 8.2 | 8 | 7.8 | 8.2 | 8.4 | 8 | 8.2 | 8.2 | 7.8 | 8.2 |
| Hexylene Glycol | 8 | 7 | 8 | 6 | 8 | 8 | — | 9 | 8 | 9 |
| Ethoxy Diglycol | 4.2 | 4 | 4.6 | 4.2 | 4.2 | 5 | 4.2 | 3 | 4.2 | 4.2 |
| Iron Oxide Particles | 0.1 | | | 0.5 | 1.0 | 3.0 | 1.0 | 0.3 | 1.0 | |
| Titanium dioxide Particles | | 5.0 | | | 4.0 | | | | 1.0 | 1.0 |
| Silicon dioxide | | | 10.0 | | | 5.0 | | 2.0 | 1.0 | 2.0 |
| Water | qs | qs | qs | qs | qs | qs | qs | qs | qs | qs |

(ii) Developer Composition

| | % Weight |
|---|---|
| Oleyl Alcohol | 0.1-2.0 |
| Steareth-21 | 1.0-5.0 |
| Acrylates Copolymer | 1.0-10.0 |
| PEG-50 | 0.1-2.0 |
| Water | 50.0-90.0 |
| Hydrogen Peroxide - 50% | 10.0 |
| Acrylates/Steareth-20 Methacrylate copolymer | 1.0 |
| Oleth-2 | 1.0 |
| Oleth-5 | 1.0 |
| Etidronic Acid | 0.05 |
| Disodium EDTA | 0.05 |
| Simethicone | 0.001 |
| Iron Oxide Particles | 0.5 |
| Zinc Oxide Particles | 3.0 |
| Carbon Black Particles | 5.0 |

(4) Hair Bleaching Composition

The hair bleaching composition part A (30 g) below was mixed with hair bleaching composition part B (45 g), then is applied to the hair and left for 30 minutes before rinsing off.

| PART A | % Weight |
|---|---|
| Potassium Persulphate | 48.0 |
| Sodium persulphate | 8.0 |
| Sodium metasilicate | 18.0 |
| Sequesterant | 1.0 |
| Ammonium chloride | 4.0 |
| Fatty chain ninionionic amphiphillic polymer: | 3.0 |
| Ser-ad FX1100 sold by Servo Delden | |
| Water soluble thickening polymer: | 2.0 |
| Sodium alginate | |
| Sodium lauryl sulphate | 3.5 |
| Calcium stearate | 2.0 |
| Polydecene | 2.0 |
| Iron Oxide Particles | 0.5 |
| Zinc Oxide Particles | 3.0 |
| Carbon Black Particles | 5.0 |

| PART B | % Weight |
|---|---|
| Cetostearyl alcohol | 2.5 |
| Trideceth-2 carboxamide MEA | 0.8 |
| 30 EO oxyethylenated cetostearyl alcohol | 0.6 |
| Pentasodium pentatate | 0.05 |
| Tetrasodium pyrophosphate | 0.03 |
| Sodium stannate | 0.02 |
| Hydrogen peroxide (50% active) | 18.0 |
| Phosphoric acid | to pH 2 |
| Water | q.s. |

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated herein by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition for application to keratin fiber, said composition comprising a discrete particle aggregate, agglomerate and combinations thereof;
wherein said aggregate and/or agglomerate have a size of from about 2 nanometers to about 1000 nanometers,
further wherein said aggregate and agglomerate comprise a chromophoric material.

2. The composition of claim 1, wherein said chromophoric material is selected from the group consisting of: metal oxides, aluminum, ceramic, cerium, copper, diamond, gold, graphite, hasteloy, indium, platinum, silicon, silver, talc, tin, zinc and zirconium carbon black, gold colloid, silver colloid, metal nano-composites, non-metal nano-composites, synthetic or naturally occurring melanin and derivatives, organic pigments and mixtures thereof.

3. The composition of claim 2, wherein said metal oxides are selected from the group consisting of: aluminum oxide, zinc oxide, cerium oxide, copper oxide, silicon oxide, zirconium oxide, titanium oxide, niobium oxide, iron oxide, doped metal oxides and combinations thereof.

4. The composition of claim 1, wherein said aggregate and agglomerate possess a size of from about 10 nanometers to about 500 nanometers.

5. The composition of claim 1, wherein said aggregate and agglomerate possess a size of from about 20 nanometers to about 200 nanometers.

6. The composition of claim 1, wherein at least about 95% of said aggregates possess a size of less than about 200 nanometers.

7. The composition of claim 1, wherein at least about 95% of said aggregates possess a size of greater than about 20 nanometers.

8. The composition according to claim 1, wherein said aggregate and agglomerate possess a mean particle size of from about 2 nanometers to about 1000 nanometers.

9. A composition comprising
a. from about 0.001% to about 10%, by weight of the entire composition, of a dye
b. from about 0.001% to about 10%, by weight of the entire composition, of a discrete particle aggregate, agglomerate and combinations thereof; and
c. the balance optional ingredients and adjuncts;
wherein said aggregate and agglomerate comprise a chromophoric material; and
further wherein said aggregate and agglomerate possesses a size of from about 2 nanometers to about 1000 nanometers.

10. The composition of claim 9, wherein said chromophoric materials are selected from the group consisting of: metal oxides, aluminium, ceramic, cerium, copper, diamond, gold, graphite, hasteloy, indium, platinum, silicon, silver, talc, tin, zinc and zirconium carbon black, gold colloid, silver colloid, metal nano-composites, non-metal nano-composites, synthetic or naturally occurring melanin and derivatives, organic pigments and mixtures thereof.

11. The composition of claim 10, wherein said metal oxides are selected from the group consisting of aluminium oxide; zinc oxide, cerium oxide, copper oxide, silicon oxide, zirconium oxide, titanium oxide, niobium oxide, iron oxide, doped metal oxides and combinations thereof.

12. The composition of claim 8, wherein said dye is selected from the group consisting of oxidative dyes, direct dyes and combinations thereof.

13. The composition of claim 8, wherein said aggregate and agglomerate possess a size of from about 10 nanometers to about 500 nanometers.

14. The composition of claim 8, wherein said aggregate and agglomerate possess a size of from about 20 nanometers to about 200 nanometers.

15. The composition of claim 8, wherein at least about 95% of said aggregates possess a size of less than about 200 nanometers.

16. The composition of claim 8, wherein at least about 95% of said aggregates possess a size of greater than about 20 nanometers.

17. A method of manufacturing discrete particle aggregate, agglomerate and combinations threreof comprising the steps of
i) providing a primary particle having a particle size greater than 1000 nm
ii) preferably milling said particles and
iii) sonicating said particles.
